(19) United States
(12) Patent Application Publication
(10) Pub. No.: US 2009/0018563 A1
(43) Pub. Date: Jan. 15, 2009

(54) STRIPPING DEVICE FOR REMOVAL OF VARICOSE VEINS

(75) Inventors: MICHAEL REDTENBACHER, Vienna (AT); Oskar A. Klampfer, Vienna (AT)

Correspondence Address: Henry M. Feiereisen;Henry M. Feiereisen, LLC, Suite 4714, 350 Fifth Avenue, New York, NY, 10118, US

(73) Assignee: E-Globe Technologies Ltd., Nicosia (CY)

(21) Appl. No.: 11/777,621

(22) Filed: Jul. 13, 2007

(51) Int. Cl. A61B 17/22

(52) U.S. Cl. 606/159

(57) ABSTRACT

A vein stripping device for removal of varicose veins including a base body and a guide cable, which is connectable to the base body and insertable into a varicose vein. Attached to the base body is at least one lens which receives incident optical light to generate light energy for cutting and severing the varicose vein. The surgery is substantially pain-free and ensures a blood-dry lumen or stripping-channel.

### BACKGROUND OF THE INVENTION

The present invention relates to a vein stripping device for removal of varicose veins.

Nothing in the following discussion of the state of the art is to be construed as an admission of prior art.

Vein stripping devices are generally used in vein stripping surgery for varicose vein removal. In the past, vein stripping has been performed exclusively by using a mechanical device which is pulled by force through the patient's leg to thereby strip the varicose major vein. However, this procedure causes undesirable side effects, such as bleeding and pain.

U.S. Pat. No. 6,858,027, issued Feb. 22, 2005, describes a vein stripping device powered by an RF-source using RF energy for coagulation. A drawback of this type of vein stripper is their limited applicability. Like any type of RF-assisted surgery, RF-powered vein stripping devices cannot be used, for example, on patients with implanted pacemakers or similar devices. Moreover, RF-powered electrical vein strippers are appropriate for the removal of varicose veins in the upper thigh only. Their use in the lower thigh is unsuitable because of potential damage to a nerve or nervus saphenous in close proximity to the major vein.

It would therefore be desirable and advantageous to provide an improved vein stripping device to obviate prior art shortcomings.

### SUMMARY OF THE INVENTION

According to one aspect of the present invention, a device for removal of a varicose vein includes a vein stripper having a lens receiving incident optical coherent light to emit light energy for cutting and severing the varicose vein.

According to another aspect of the present invention, a vein stripping device for removal of varicose veins includes a base body, a guide cable, connectable to the base body and insertable into a varicose vein, and a lens arrangement having at least one lens attached to the base body and receiving incident optical coherent light to emit light energy for cutting and severing the varicose vein.

A vein stripper according to the present invention is applicable for a wide range of applications. As light energy is only emitted directly towards the front of the vein stripping device, surrounding tissue remains unaffected and is not damaged by heat. Thus, the vein stripper can also be used for surgery in the lower thigh.

The intended source of energy to be used with the vein stripping device according to the present invention involves an external source of coherent light, such as a laser generator.

According to another feature of the present invention, the light energy can be conducted to the stripper head by means of at least one optical cable or light guide, which can be securely fixed to the stripper head or through intervention of an optical connector.

According to another feature of the present invention, the base body has an end surface formed with an opening for passage of the guide cable, and a plurality of lenses may be provided which are arranged about the opening so as to surround the guide cable in coaxial relationship, for executing the cutting and coagulation operations during surgical vein stripping procedures. The provision of several lenses assures a cutting of the venous side branches and safe and reliable suturing by coagulation. The energy emitted from the lenses is able to heat up side branches of a major vein to be extracted. These side branches are first severed, or cut, by the light energy from the source of coherent light and then sutured by the same type of energy from the lens used for cutting, or from another lens in close proximity thereto.

The vein stripping device functions with various sources of coherent light, i.e. with various types of laser generators. In order to efficiently operate the vein stripping device and to consume only minimal energy, the wavelength of a laser source should be relatively high, as the “water absorption coefficient” of tissue increases at longer wavelength. Utilization of a relatively high wavelength will also result in a shallow tissue penetration of the light rays. This is especially useful in connection with a surgery within the lower thigh. Suitably, the laser source is a Holium-YAG laser source or a CO₂ laser source, which emits light at a wavelength of approximately 2 μm or longer. Currently preferred is a wavelength of about 3 micron.

In the event, more than one lens is used, it also possible to use different light sources for severing and suturing the venous side branches. The use of Holium-YAG and CO₂ lasers, as different light sources for severing and suturing the venous side-branches is currently preferred.

Suitably, the coherent light is dispersed circularly all around a length axis of the base body from the lenses. The direction of dispersion is mainly “forward”, i.e. in parallel relationship to the direction the stripper, is being pulled through the leg of the patient.

The lenses may be constructed of different type so as to emit light energy with minimal losses and as evenly as possible about the perimeter of the lens.

According to another feature of the present invention, the lens may be implemented by an extension of the light guide through winding the extension at least once about an end surface of the base body, with the extension of the optical light guide provided with irregularities in the form of multiple microbends. A “microbend” constitutes a sharp curvature involving a local axial displacement (from the axis of the light guide itself) of a few micrometers, thereby causing a spatial deviation of the wavelength of a few millimeters. While a microbend is generally considered a defect in a light guide, the presence of microbends in accordance with present invention is now intentionally applied evenly along the surface of the exposed light guide at the front surface of the stripping device. The end of the light guide can be wound once or several times about the front surface of the vein stripper, with the microbends being provided such as to radiate the light energy to the front as evenly as possible.

The lens on the front end of the base body of the vein stripping device covers a full 360° circle about the length-axis of the base body. The lens may be circular, elliptical, or have any other shape so long a closed loop, or almost closed loop, is realized.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1A is an exploded side view of one embodiment of a vein stripping device according to the invention;

FIG. 1B is a side view of the vein stripping device in assembled state;

FIG. 2 is a perspective view of a stripper head of the vein stripping device according to the present invention;

FIG. 3 is a perspective view of the stripper head of the vein stripping device with an exemplary vein to be extracted;

FIG. 4 is a schematic view of a lens arrangement of the vein stripping device;

FIG. 5 is a schematic view of a light guide for conducting light to the lens arrangement of the vein stripping device, and

FIG. 6 is an enlarged detailed view of the area encircled in FIG. 5 and marked VI.

### DETAILED DESCRIPTION OF PREFERRED EMBODIMENTS

Throughout all the Figures, same or corresponding elements may generally be indicated by same reference numerals. These depicted embodiments are to be understood as illustrative of the invention and not as limiting in any way. It should also be understood that the figures are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

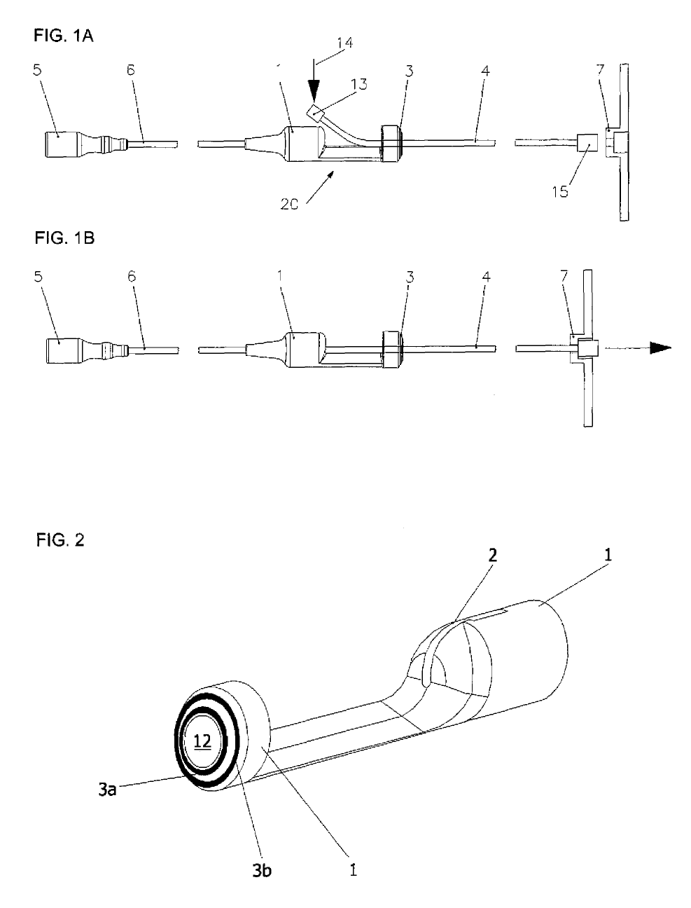

Turning now to the drawing, and in particular to FIG. 1A, there is shown an exploded side view of one embodiment of a vein stripping device according to the invention, including a stripper head, shown in midsection of FIG. 1A, and generally designated by reference numeral 20. The stripper head 20 includes a base body 1 which, as shown in FIG. 2, is formed approximately in midsection with a slot 2 (not visible in FIG. 1A). Located on the front end of the base body 1 is a lens arrangement, generally designated by reference numeral 3. The lens arrangement 3 may include one or more individual lenses, indicated by reference numerals 3a, 3b. Although the drawing shows only the presence of two such lenses, it will be appreciated by persons skilled in the art that the lens arrangement may, of course, include more than two lenses or only just one lens.

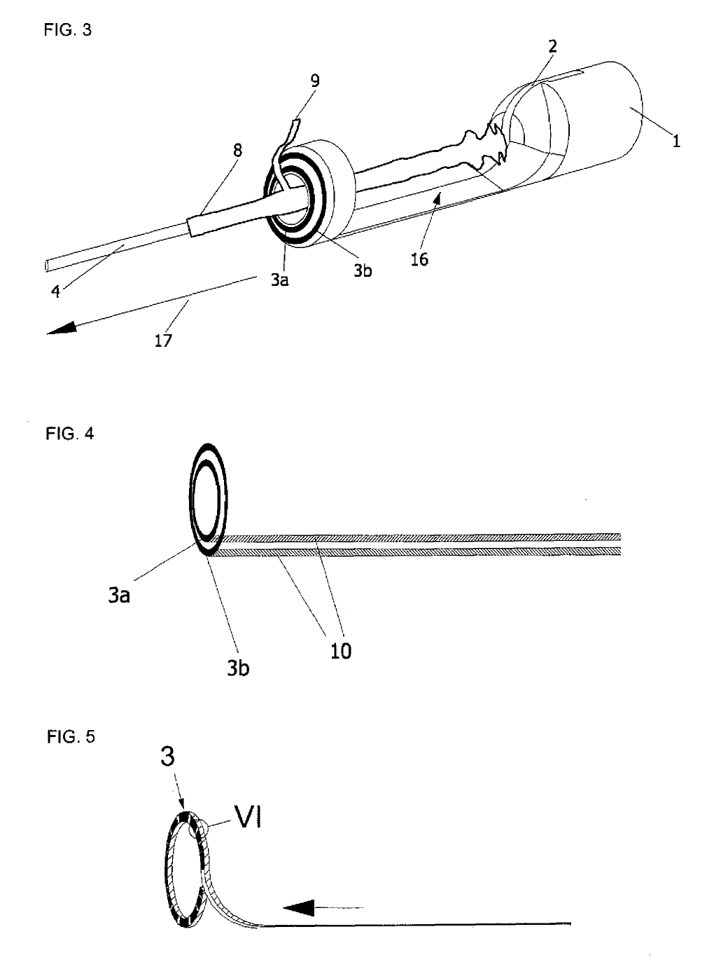

To the left of the stripper head 20 is a fiber-optic connector 5 which has one end connected with a fiber-optic cable 6 by which light generated by an unillustrated external light source is conducted. The connector 5 is hereby constructed for attachment to the light source, e.g. a laser source such as a Holium-YAG laser or a CO₂ laser. The other end of the fiber optic cable 6 is connected to the base body 1 of the stripper head 20. The base body 1 has a central opening 12 (FIG. 2) for passage of a guide cable 4 which is fed through a vein 8 (FIG. 3) to be extracted. The guide cable 4 has one end formed with an olive 13 for acceptance in the slot 2 in order to connect the guide cable 4 to the base body 1, as indicated by arrow 14. The other end of the guide cable 4 is also formed with an olive 15 for acceptance in a pulling handle 7. The assembled state of the vein stripping device is shown in FIG. 1B.

The stripper head 20 is shown in greater detail, on an enlarged scale, in FIG. 2, depicting the two circular lenses 3a, 3b in concentric surrounding relationship about the opening 12 at the front surface of the base body 1. The base body 1 is typically made of plastics but, of course, may also be made of ceramic or other suitable material.

Referring now to FIG. 3, there is shown a perspective view of the stripper head 20 during surgical procedure. The guide cable 4 is connected to the base body 1 via the slot 2 and inserted in a vein 8 to be removed. Reference numeral 9 designates a typical branch which is about to be severed. As the guide cable 4 moves within the vein 8, the vein 8 folds up much like a bellows within the “storage area” 16 in midsection of the stripper head 20. The direction of pull of the vein stripping device by means of the guide cable 4 is indicated by arrow 17.

FIG. 4 shows a schematic detailed view of the lens arrangement 3 on the front surface of the base body 1. The lenses 3a, 3b receive coherent light via fiber-optic cables or light guides 10. Of course, coherent light energy can be conducted to the front of the stripper head 20 in other ways as well and dispersed there.

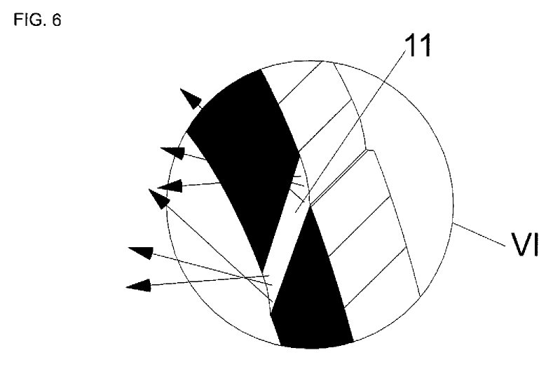

Turning now to FIG. 5, there is shown a currently preferred light guide 10 for conducting light to the lens arrangement 3. The light guide 10 is hereby provided with microbends 11 (FIG. 6) and has a rectangular cross-section (e.g. approximately 2 mm wide and 0.5 mm thick). The light guide 10 is routed from the connector 5 to the front of the stripper head 20 and then wound once, or several times, about the length axis of the base body 1. In the region of this loop at the front surface of the stripper head 20, numerous very small defects to the surface, called “clinks” or “microbends”, are induced into the light guide 10 by means of precision crimping. Each such microbend 11 causes a slight light attenuation, or loss, within the light guide 10, and coherent light scatters at the position of the microbend 11 as a result of this surface defect. These numerous defects are provided of controlled size and evenly distributed along the entire length of the light guide 10 that has been wound about the front surface of the stripper head 20. As a consequence, coherent light and resultant light energy, is evenly distributed about the front surface to thereby ensure a cutting and coagulation of the vein 8 to be removed.

FIG. 6 is an enlarged detailed view of the area encircled in FIG. 5 and marked VI, to depict a single such microbend 11 to intentionally induce such a defect to the surface of the light guide 10. The displacement is, of course, shown exaggerated, to indicate where and in which direction light is scattered by a microbend 11. In reality, the axial displacement within the light guide 10 is only a few microns.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

For example, when using a multi-fiber light guide, the lens may be constructed in such a way that the ends of the individual fibers are arranged about the front surface of the base body 1. Also other types of lens arrangements may be conceivable as well.

What is claimed as new and desired to be protected by Letters Patent is set forth in the appended claims and includes equivalents of the elements recited therein:

What is claimed is:

1. A device for removal of a varicose vein, comprising a vein stripper having an optical lens receiving incident optical coherent light to generate light energy for cutting and severing the varicose vein.

2. A vein stripping device for removal of a varicose vein, comprising:
a base body;
a guide cable, connectable to the base body and insertable into a varicose vein; and
a lens arrangement having at least one lens attached to the base body and receiving incident optical coherent light to emit light energy for cutting and severing the varicose vein.

3. The vein stripping device of claim 1, wherein the base body has an end surface formed with an opening for passage of the guide cable, said lens arrangement having a plurality of lenses arranged about the opening so as to surround the guide cable in coaxial relationship.

4. The vein stripping device of claim 1, wherein the base body has a slot, said guide cable being constructed for attachment in the slot.

5. The vein stripping device of claim 1, further comprising an optical light guide for conducting the light energy to the lens.

6. The vein stripping device of claim 5, wherein the optical light guide has a rectangular cross-section.

7. The vein stripping device of claim 6, wherein the optical light guide has, a width of approximately 2 mm and a thickness of approximately 0.5 mm.

8. The vein stripping device of claim 5, wherein the optical light guide has a base body-proximal end for securement to the base body.

9. The vein stripping device of claim 5, further comprising an optical connector mounted to the light guide on a base body-distal end thereof and an optical light source emitting coherent light and connectable to the light guide via the connector.

10. The vein stripping device of claim 9, wherein the light source is a laser source.

11. The vein stripping device of claim 10, wherein the laser source is a Holium-YAG laser or a CO₂ laser.

12. The vein stripping device of claim 10, wherein the laser source emits light at a wavelength of approximately 2 μm or longer.

13. The vein stripping device of claim 10, wherein the laser source emits light at a wavelength of 3 μm.

14. The vein stripping device of claim 3, further comprising a plurality of different light sources for emitting optical light to the lenses in one-to-one correspondence.

15. The vein stripping device of claim 5, wherein the lens is formed by an extension of the light guide through winding the extension at least once about an end surface of the base body, said extension of the optical light guide being provided with multiple microbends.

16. The vein stripping device of claim 2, wherein the lens is constructed in the form of a closed or roughly closed loop.

17. The vein stripping device of claim 14, wherein the loop has a circular or elliptical configuration.

18. The vein stripping device of claim 14, wherein the lens includes a plurality of microbends induced upon the extension of the light guide and disposed in the loop about a longitudinal axis of the base body for dispersing incident coherent light circularly and in parallel relationship to a movement direction of the guide cable.
